(19) United States
(12) Patent Application Publication
(10) Pub. No.: US 2024/0185962 A1
(43) Pub. Date: Jun. 6, 2024

(54) AQUEOUS COMPOSITION SENSORIAL IMPACT DETERMINATION METHOD, AQUEOUS COMPOSITION INGREDIENT QUANTITY DETERMINATION METHOD AND CORRESPONDING SYSTEMS

(71) Applicant: Firmenich SA, Satigny (CH)

(72) Inventors: Wolfgang FIEBER, Satigny (CH); Resti MAGTOTO, Plainsboro, NJ (US)

(21) Appl. No.: 18/553,520

(22) PCT Filed: Apr. 29, 2022
(86) PCT No.: PCT/EP2022/061432

§ 371 Date: Sep. 29, 2023

Related U.S. Application Data
(60) Provisional application No. 63/181,630, filed on Apr. 29, 2021.

(30) Foreign Application Priority Data
Jun. 9, 2021 (EP) 21173395.1

(51) Int. Cl. G16C 20/30; G16C 60/00

(52) U.S. Cl. CPC G16C 20/30; G16C 60/00

(57) ABSTRACT

The aqueous composition sensorial impact determination method (100), comprises: —a step (105) of inputting at least one fragrant molecule digital identifier, —a step (106) of associating a value representative of a quantity of the associated fragrant molecule to be input, —a step (107) of inputting at least one surfactant molecule digital identifier —a step (110) of computing, by a computing device, a relative concentration of at least one fragrant molecule of the formula in the water phase and in the micellar phase, —a step (115) of retrieving, by a computing device, a liquid-gas partition coefficient of at least one said fragrant molecule, —a step (120) of computing a gas phase concentration of at least one said fragrant molecule, —a step (125) of estimating a psychophysical sensorial intensity for at least one fragrant molecule and —step (130) of outputting the psychophysical sensorial intensity for at least one fragrant molecule of the formula.

### TECHNICAL FIELD OF THE INVENTION

The present invention relates to an aqueous composition sensorial impact determination method, an aqueous composition ingredient quantity determination method and the corresponding systems. This invention can be applied to the general field of perfumery at large and in particular related to personal care and home care applications.

### BACKGROUND OF THE INVENTION

Bloom is commonly referred to as the sensorial impact of a fragrance after dilution of an aqueous surfactant-based application for personal care and home care. Blooming is responsible for the pleasantness for example of a liquid hand soap, a shower gel, a shampoo, or a hard surface cleaner. As such, this parameter has been investigated over the years as a criterion for likeability of such applications. The capacity to predict how a particular perfume will behave once a personal care or a home care application is in contact with water is key for more efficient design and evaluation process in this space.

Traditional methods, based upon empirical experimentation, would revolve around designing and producing a formula in a particular application, having a subject test said formula in a controlled simulation environment (such as a shower cabin, for example), and then having the subject take a survey to evaluate how the particular bloom of the formula was perceived. The outcome of such surveys was used, in turn, to redesign and upgrade the formula.

Modern approaches, such as the one disclosed in patent U.S. Pat. No. 9,364,409, disclose combinations of surfactant molecule and perfume that provide a defined sensorial performance. In particular, odor intensity scores (OIS) are described that provide information about bloom efficiency of a given combination of perfume and surfactant molecule. Such approaches are limited in that they do not account for the relative proportions of surfactant molecule and perfume, nor do they account for the interaction of the particular application with water and/or within the airspace.

Other modern approaches, such as the one disclosed in patent application US 2007/0071780 relate to personal care applications having an efficient perfume bloom. Combinations of surfactant molecule comprising a perfume booster accord. These booster accords are defined by a low ODT (odor detection threshold) and a high “Human recognition slope factor (HRSF)”. However, such approaches are limited in that they do not account for the interactions between the fragrant ingredients and the surfactant in the application, nor do they account for the interaction of the particular application with water and/or within the airspace.

As such, there exists no current satisfying system to model the bloom of an aqueous fragrant composition, leading to increased application design time and cost.

### SUMMARY OF THE INVENTION

The present invention is intended to remedy all or part of these disadvantages.

To this effect, according to a first aspect, the present invention aims at an aqueous composition sensorial impact determination method, comprising:
a step of inputting at least one fragrant molecule digital identifier, upon a computer interface, said input defining a formula,
a step of associating, for at least one input fragrant molecule digital identifier, a value representative of a quantity of the associated fragrant molecule to be input,
a step of inputting at least one surfactant molecule digital identifier, upon a computer interface, said identifier being representative of a surfactant molecule in which the input surfactant molecule is organized in micelles, and where the input fragrant molecule partitions between the aqueous phase and the micellar phase of the surfactant molecule,
a step of computing, by a computing device, a relative concentration of at least one fragrant molecule of the formula in the water phase and in the micellar phase formed by the corresponding surfactant as a function of the input formula and the associated quantity for at least one fragrant molecule digital identifier and the input surfactant molecule digital identifier,
a step of retrieving, by a computing device, a liquid-gas partition coefficient of at least one said fragrant molecule,
a step of computing, by a computing device, a gas phase concentration of at least one said fragrant molecule as a function of the liquid-gas partition coefficient and of the relative concentration in the water phase of said fragrant molecule,
a step of estimating, by a computing device, a psychophysical sensorial intensity for at least one fragrant molecule of the formula as a function of the computed gas phase concentration and
a step of outputting, upon a computer interface, the psychophysical sensorial intensity for at least one fragrant molecule of the formula.

Such provisions allow for the accurate modeling of the base-perfume interactions as well as subsequent liquid-gas phase interactions, gas phase concentrations and ultimately perceived intensity of bloom. Such a model allows for more dynamic and modular considerations during perfume design, limiting the cost and time of such a step.

Such embodiments allow modeling key parameters for bloom experience such as dilution with water, specific time delays, and definition of environment and defining applications bases (i.e., surfactant molecules).

In particular embodiments, the method object of the present invention further comprises a step of setting, upon a computer interface, values of sensory evaluation parameters representative of at least one of:
temperature of the water or the air,
liquid volume of aqueous composition,
air volume into which the fragrant molecule is transferred,
application surface of the aqueous composition and evolution over time,
dilution factor,
rate of addition of water,
agitation of aqueous phase,
ambient air flow and/or
time intervals or total duration, such values being used at least one of the steps upstream of the step of outputting.

Such embodiments allow for the more accurate prediction of bloom performance in a given environment. This allows perfume design optimization as a function of the environment characteristics in which this perfume is meant to be used.

In particular embodiments, the step of computing, by a computing device, a gas phase concentration is performed as a function of time, the psychophysical sensorial intensity estimated being determined as a function of said gas phase concentration.

Such embodiments allow for the prediction of the behavior, over time, of a perfume.

In particular embodiments, the step of computing a relative concentration is performed using the equation:

K_{M}=AF·P_{O/W}

where:
K_{M} is the micelle-water partition coefficient of the fragrant molecules between micellar and aqueous phases,
AF is an affinity factor and
P_{O/W} represents the octanol-water partition coefficient.

Such embodiments allow for the accurate modeling of the part of the application that indeed contributes to blooming.

In particular embodiments, the method object of the present invention further comprises, upstream of the step of computing a relative concentration:
a step of self-diffusion NMR spectroscopy of at least one aqueous fragrant molecule and at least one surfactant molecule, and
step of inscription, within a computer memory, of a calculated affinity factor value for each said surfactant molecule,
   the step of computing a relative concentration being performed as a function of at least one affinity factor value stored within the computer memory.

Such embodiments allow for the creation of a database of affinity factor values that allow for a more accurate modeling of the bloom phenomenon.

In particular embodiments, the method object of the present invention further comprises a step of determination, by a computing device, of an evaluation parameter as a function of a value representative of time since contact between the aqueous composition and a stream of water, the step of computing a gas phase concentration being performed as a function of the evaluation parameter determined.

Such embodiments allow for the accurate modeling of the evolution of spread of the application over the body or hair or onto a surface and the impact of that area on the blooming phenomenon.

In particular embodiments, the method object of the present invention further comprises a step of replacing, by a computing device, at least one fragrant molecule digital identifier in the input formula as a function of the estimated psychophysical sensorial intensity of each said ingredient and the estimated psychophysical sensorial intensity of at least one other fragrant molecule.

Such embodiments allow for the dynamic replacement, or suggestion of replacement, of an ingredient in a formula by another ingredient based upon the bloom performance of said ingredients.

In particular embodiments, the method object of the present invention further comprises a step of defining, upon a computer interface, a psychophysical sensorial intensity threshold for at least one determined fragrant molecule digital identifier, the step of replacing being performed as a function of the determined threshold.

Such embodiments allow for the selection of alternative ingredients as a function of the defined threshold.

In particular embodiments, the method object of the present invention further comprises a step of calculation, by a computing device, of a psychophysical sensorial intensity evolution function of a fragrant molecule as a function of:
the gas phase concentration of said fragrant molecule and
a characteristic psychophysical sensorial intensity dose response curve linking gas phase concentration to psychophysical sensorial intensity,
   the step of replacing being performed as a function of the psychophysical sensorial intensity evolution function of a fragrant molecule configured to be replaced and/or to replace another fragrant molecule in the formula.

Such embodiments allow for the selection of alternative ingredients as a function of the capacity of said ingredients to increase their blooming performance if their relative concentration increases. Such a parameter offers more perfume design flexibility.

In particular embodiments, the method object of the present invention further comprises a step of determining, by a computing device, a value representative of a sensitivity of variation of gas phase concentration at a reference point in the characteristic psychophysical sensorial intensity dose response curve of a fragrant molecule, the step of replacing being performed as a function of the sensibility of a fragrant molecule configured to be replaced and/or to replace another fragrant molecule in the formula.

According to a second aspect, the present invention aims at an aqueous composition ingredient quantity determination method, comprising:
a means for inputting at least one fragrant molecule digital identifier, upon a computer interface, said input defining a formula,
a means for inputting at least one surfactant molecule digital identifier, upon a computer interface, said identifier being representative of a surfactant molecule in which the input surfactant molecule is organized in micelles, and where the input fragrant molecule partitions between the aqueous phase and the micellar phase of the surfactant molecule,
a step of defining, upon a computer interface, a value of target psychophysical sensorial intensity for at least one fragrant molecule of the formula,
a step of estimating, by a computing device, a gas phase concentration for at least one fragrant molecule of the formula as a function of the defined target psychophysical sensorial intensity,
a step of computing, by a computing device, a liquid-phase concentration of at least one said fragrant molecule as a function of the estimated gas phase concentration of said fragrant molecule,
a step of computing, by a computing device, a relative concentration of at least one fragrant molecule of the formula in the water phase and in the micellar phase formed by the corresponding surfactant as a function of the liquid-phase concentration computed and
a step of outputting, upon a computer interface, the relative concentration of at least one fragrant molecule of the formula.

These provisions allow for reverse perfume design in which the ingredients are selected as a function of the desired bloom performance and/or in which the relative concentration of these ingredients is determined as function of said performance.

In particular embodiments, the method object of the present invention further comprises a step of assembling the formula resulting from said method.

Such embodiments allow for the materialization of the input formula, the generated formula or the modified formula.

According to a third aspect, the present invention aims at an aqueous composition sensorial impact determination system, comprising:
a means for inputting at least one fragrant molecule digital identifier, upon a computer interface, said input defining a formula,
a means of associating, for at least one input fragrant molecule digital identifier, a value representative of a quantity of the associated fragrant molecule to be input,
a means for inputting at least one surfactant molecule digital identifier, upon a computer interface, said identifier being representative of a surfactant molecule in which the input surfactant molecule is organized in micelles, and where the input fragrant molecule partitions between the aqueous phase and the micellar phase of the surfactant molecule,
a means for computing, by a computing device, a relative concentration of at least one fragrant molecule of the formula in the water phase and in the micellar phase formed by the corresponding surfactant as a function of the input formula and the associated quantity for at least one fragrant molecule digital identifier and the input surfactant molecule digital identifier,
a means for retrieving a liquid-gas partition coefficient of at least one said fragrant molecule,
a means for computing a gas phase concentration of at least one said fragrant molecule as a function of the liquid-gas partition coefficient and of the relative concentration in the water phase of said fragrant molecule,
a means for estimating a psychophysical sensorial intensity for at least one fragrant molecule of the formula as a function of the computed gas phase concentration and
a means for outputting, upon a computer interface, the psychophysical sensorial intensity for at least one fragrant molecule of the formula.

The benefits of this system are similar to the benefits of the corresponding method.

According to a fourth aspect, the present invention aims at an aqueous composition ingredient quantity determination system, comprising:
a means for inputting at least one fragrant molecule digital identifier, upon a computer interface, said input defining a formula,
a means for inputting at least one surfactant molecule digital identifier, upon a computer interface, said identifier being representative of a surfactant molecule in which the input surfactant molecule is organized in micelles, and where the input formula partitions between the aqueous phase and the micellar phase of the surfactant molecule,
a means for defining, upon a computer interface, a value of target psychophysical sensorial intensity for at least one fragrant molecule of the formula,
a means for estimating, by a computing device, a gas phase concentration for at least one fragrant molecule of the formula as a function of the defined target psychophysical sensorial intensity,
a means for computing, by a computing device, a liquid-phase concentration of at least one said fragrant molecule as a function of the estimated gas phase concentration of said fragrant molecule,
a means for computing, by a computing device, a relative concentration of at least one fragrant molecule of the formula in the water phase and in the micellar phase formed by the corresponding surfactant as a function of the liquid-phase concentration computed and
a means for outputting, upon a computer interface, the relative concentration of at least one fragrant molecule of the formula.

The benefits of this system are similar to the benefits of the corresponding method.

### BRIEF DESCRIPTION OF THE DRAWINGS

Other advantages, purposes and particular characteristics of the invention shall be apparent from the following non-exhaustive description of at least one particular methods and systems object of this invention, in relation to the drawings annexed hereto, in which:

FIG. 1 represents, schematically, a first particular succession of steps of the method subject of the present invention,

FIG. 2 represents, schematically, a second particular succession of steps of the method subject of the present invention,

FIG. 3 represents, schematically, a first particular embodiment of the system subject of the present invention and

FIG. 4 represents, schematically, a second particular embodiment of the system subject of the present invention.

### DETAILED DESCRIPTION OF THE INVENTION

This description is not exhaustive, as each feature of one embodiment may be combined with any other feature of any other embodiment in an advantageous manner. Also, various inventive concepts may be embodied as one or more methods, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

The indefinite articles ‘a’ and ‘an’, as used herein in the specification and in the claims, unless clearly indicated to the contrary, should be understood to mean ‘at least one’.

The phrase ‘and/or’, as used herein in the specification and in the claims, should be understood to mean ‘either or both’ of the elements so conjoined, i.e. elements that are conjunctively present in some cases and disjunctively present in other cases. Multiple elements listed with ‘and/or’ should be construed in the same fashion, i.e. ‘one or more’ of the elements so conjoined. Other elements may optionally be present other than the elements specifically identified by the ‘and/or’ clause whether related or unrelated to those elements specifically identified. Thus, as a non-limiting example, a reference to ‘A and/or B’, when used in conjunction with open-ended language such as ‘comprising’ can refer, in one embodiment, to A only (optionally including elements other than B); in another embodiment, to B only (optionally including elements other than A); in yet another embodiment, to both A and B (optionally including other elements); etc.

As used herein in the specification and in the claims, ‘or’ should be understood to have the same meaning as ‘and/or’ as defined above. For example, when separating items in a list, ‘or’ or ‘and/or’ shall be interpreted as being inclusive, i.e. the inclusion of at least one, but also including more than one, of a number or list of elements, and, optionally, additional unlisted items. Only terms clearly indicated to the contrary, such as ‘only one of’ or ‘exactly one of’, or, when used in the claims, ‘consisting of’, will refer to the inclusion of exactly one element of a number or list of elements. In general, the term ‘or’ as used herein shall only be interpreted as indicating exclusive alternatives (i.e. ‘one or the other but not both’) when preceded by terms of exclusivity, such as ‘either,’ ‘one of,’ ‘only one of’, or ‘exactly one of’. ‘Consisting essentially of,’ when used in the claims, shall have its ordinary meaning as used in the field of patent law.

As used herein in the specification and in the claims, the phrase ‘at least one’, in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase ‘at least one’ refers, whether related or unrelated to those elements specifically identified. Thus, as a non-limiting example, ‘at least one of A and B’ (or, equivalently, ‘at least one of A or B’, or, equivalently ‘at least one of A and/or B’) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as ‘comprising,’ ‘including,’ ‘carrying,’ ‘having,’ ‘containing,’ ‘involving,’ ‘holding,’ ‘composed of’, and the like are to be understood to be open-ended, i.e. to mean including but not limited to. Only the transitional phrases ‘consisting of’ and ‘consisting essentially of’ shall be closed or semi-closed transitional phrases, respectively.

It should be noted at this point that the figures are not to scale.

The content of U.S. Ser. No. 62/911,096 is incorporated herein by reference.

As used herein, the terms “fragrant molecule” designate any molecule, preferably presenting a flavoring or fragrance capacity, that is activating the odorant receptors of animals and preferably humans. In other words, by “fragrant molecule” it is meant here a compound, which is used in a perfuming preparation or a composition to impart a hedonic effect, i.e., used for the primary purpose of conferring or modulating an odor. In other words, such a co-ingredient, to be considered as being a perfuming one, must be recognized by a person skilled in the art as being able to impart or modify in a positive or pleasant way the odor of a composition, and not just as having an odor. The terms “compound” or “ingredient” designate the same items as “fragrant molecule”. Fragrant molecules are also known as perfumery raw materials (PRM). The nature and type of the fragrant ingredients present in the base do not warrant a more detailed description here, which in any case would not be exhaustive, the skilled person being able to select them on the basis of his general knowledge and according to the intended use or application and the desired organoleptic effect. In general terms, these fragrant ingredients belong to chemical classes as varied as alcohols, lactones, aldehydes, ketones, esters, ethers, acetates, nitriles, terpenoids, nitrogenous or sulphurous heterocyclic compounds and essential oils, and said fragrant ingredients can be of natural or synthetic origin. Example of fragrant ingredients are listed in reference texts such as the book by S. Arctander, Perfume and Flavor Chemicals, 1969, Montclair, New Jersey, USA, or its more recent versions, or in other works of a similar nature, as well as in the abundant patent literature in the field of perfumery.

The term “formula” designates a liquid, solid or gaseous assembly of at least one fragrant molecule. The formula may further comprise at least one perfumery carrier and/or at least one perfumery adjuvant.

By “perfumery carrier” it is meant here a material which is practically neutral from a perfumery point of view, i.e., that does not significantly alter the organoleptic properties of perfuming ingredients. Said carrier may be a liquid or a solid.

As liquid carrier one may cite, as non-limiting examples, an emulsifying system, i.e., a solvent and a surfactant system, or a solvent commonly used in perfumery. A detailed description of the nature and type of solvents commonly used in perfumery cannot be exhaustive. However, one can cite as non-limiting examples, solvents such as butylene or propylene glycol, glycerol, dipropyleneglycol and its monoether, 1,2,3-propanetriyl triacetate, dimethyl glutarate, dimethyl adipate 1,3-diacetyloxypropan-2-yl acetate, diethyl phthalate, isopropyl myristate, Abalyn® (rosin resins, available from Eastman), benzyl benzoate, benzyl alcohol, 2-(2-ethoxyethoxy)-1-ethanol, tri-ethyl citrate or mixtures thereof, which are the most commonly used or also naturally derived solvents like glycerol or various vegetable oils such as palm oil, sunflower oil or linseed oil. For the compositions which comprise both a perfumery carrier and a perfumery base, other suitable perfumery carriers than those previously specified, can be also ethanol, water/ethanol mixtures, limonene or other terpenes, isoparaffins such as those known under the trademark Isopar® (origin: Exxon Chemical) or glycol ethers and glycol ether esters such as those known under the trademark Dowanol® (origin: Dow Chemical Company), or hydrogenated castors oils such as those known under the trademark Cremophor@ RH 40 (origin: BASF).

Solid carrier is meant to designate a material to which the perfuming composition or some element of the perfuming composition can be chemically or physically bound. In general such solid carriers are employed either to stabilize the composition, or to control the rate of evaporation of the compositions or of some ingredients. Solid carriers are of current use in the art and a person skilled in the art knows how to reach the desired effect. However by way of non-limiting examples of solid carriers, one may cite absorbing gums or polymers or inorganic materials, such as porous polymers, cyclodextrines, dextrines, maltodextrines wood based materials, organic or inorganic gels, clays, gypsum talc or zeolites.

As other non-limiting examples of solid carriers, one may cite encapsulating materials. Examples of such materials may comprise wall-forming and plasticizing materials, such as glucose syrups, natural or modified starches, hydrocolloids, cellulose derivatives, polyvinyl acetates, polyvinylalcohols, proteins or pectins, plant gums such as acacia gum (Gum Arabic), urea, sodium chloride, sodium sulphate, zeolite, sodium carbonate, sodium bicarbonate, clay, talc, calcium carbonate, magnesium sulfate, gypsum, calcium sulfate, magnesium oxide, zinc oxide, titanium dioxide, calcium chloride, potassium chloride, magnesium chloride, zinc chloride, carbohydrates, saccharides such as sucrose, mono-, di-, and polysaccharides and derivatives such as chitosan, starch, cellulose, carboxymethyl methylcellulose, methylcellulose, hydroxyethyl cellulose, ethyl cellulose, propyl cellulose, polyols/sugar alcohols such as sorbitol, maltitol, xylitol, erythritol, and isomalt, polyethylene glycol (PEG), polyvinyl pyrrolidin (PVP), polyvinyl alcohol, acrylamides, acrylates, polyacrylic acid and related, maleic anhydride copolymers, amine-functional polymers, vinyl ethers, styrenes, polystyrenesulfonates, vinyl acids, ethylene glycol-propylene glycol block copolymers, vegetable gums, gum acacia, pectins, xanthanes, alginates, carragenans, citric acid or any water soluble solid acid, fatty alcohols or fatty acids and mixtures thereof, or yet the materials cited in reference texts such as H. Scherz, Hydrokolloide: Stabilisatoren, Dickungs- und Geliermittel in Lebensmitteln, Band 2 der Schriftenreihe Lebensmittelchemie, Lebensmittelqualität, Behr's Verlag Gmbh & Co., Hamburg, 1996. The encapsulation is a well-known process to a person skilled in the art, and may be performed, for instance, by using techniques such as spray-drying, agglomeration or yet extrusion; or consists of a coating encapsulation, including coacervation and complex coacervation techniques.

As non-limiting examples of solid carriers, one may cite in particular the core-shell capsules with resins of aminoplast, polyamide, polyester, polyurea or polyurethane type or a mixture threof (all of said resins are well known to a person skilled in the art) using techniques like phase separation process induced by polymerization, interfacial polymerization, coacervation or altogether (all of said techniques have been described in the prior art), optionally in the presence of a polymeric stabilizer or of a cationic copolymer.

Resins may be produced by the polycondensation of an aldehyde (e.g. formaldehyde, 2,2-dimethoxyethanal, glyoxal, glyoxylic acid or glycolaldehyde and mixtures thereof) with an amine such as urea, benzoguanamine, glycoluryl, melamine, methylol melamine, methylated methylol melamine, guanazole and the like, as well as mixtures thereof. Alternatively one may use preformed resins alkylolated polyamines such as those commercially available under the trademark Urac® (origin: Cytec Technology Corp.), Cymel® (origin: Cytec Technology Corp.), Urecoll® or Luracoll® (origin: BASF).

Other resins are the ones produced by the polycondensation of a polyol, like glycerol, and a polyisocyanate, like a trimer of hexamethylene diisocyanate, a trimer of isophorone diisocyanate or xylylene diisocyanate or a Biuret of hexamethylene diisocyanate or a trimer of xylylene diisocyanate with trimethylolpropane (known with the tradename of Takenate@, origin: Mitsui Chemicals), among which a trimer of xylylene diisocyanate with trimethylolpropane and a Biuret of hexamethylene diisocyanate are preferred.

Some of the seminal literature related to the encapsulation of perfumes by polycondensation of amino resins, namely melamine-based resins with aldehydes includes articles such as those published by K. Dietrich et al. Acta Polymerica, 1989, vol. 40, pages 243, 325 and 683, as well as 1990, vol. 41, page 91. Such articles already describe the various parameters affecting the preparation of such core-shell microcapsules following prior art methods that are also further detailed and exemplified in the patent literature. U.S. Pat. No. 4,396,670 to the Wiggins Teape Group Limited is a pertinent early example of the latter. Since then, many other authors have enriched the literature in this field and it would be impossible to cover all published developments here, but the general knowledge in encapsulation technology is very significant. More recent publications of pertinence, which disclose suitable uses of such microcapsules, are represented for example by the article of K. Bruyninckx and M. Dusselier, ACS Sustainable Chemistry & Engineering, 2019, vol. 7, pages 8041-8054.

By “perfumery adjuvant”, it is meant here an ingredient capable of imparting additional added benefit such as a color, a particular light resistance, chemical stability, etc. A detailed description of the nature and type of adjuvant commonly used in perfuming composition cannot be exhaustive, but it has to be mentioned that said ingredients are well known to a person skilled in the art. One may cite as specific non-limiting examples the following: viscosity agents (e.g. surfactants, thickeners, gelling and/or rheology modifiers), stabilizing agents (e.g. preservatives, antioxidant, heat/light and or buffers or chelating agents, such as BHT), coloring agents (e.g. dyes and/or pigments), preservatives (e.g. antibacterial or antimicrobial or antifungal or anti irritant agents), abrasives, skin cooling agents, fixatives, insect repellants, ointments, vitamins and mixtures thereof. By “fixative” also called “modulator”, it is understood here an agent having the capacity to affect the manner in which the odour, and in particular the evaporation rate and intensity, of the compositions incorporating said modulator can be perceived by an observer or user thereof, over time, as compared to the same perception in the absence of the modulator. In particular, the modulator allows prolonging the time during which their fragrance is perceived. Non-limiting examples of suitable modulators may include methyl glucoside polyol; ethyl glucoside polyol; propyl glucoside polyol; isocetyl alcohol; PPG-3 myristyl ether; neopentyl glycol diethylhexanoate; sucrose laurate; sucrose dilaurate, sucrose myristate, sucrose palmitate, sucrose stearate, sucrose distearate, sucrose tristearate, hyaluronic acid disaccharide sodium salt, sodium hyaluronate, propylene glycol propyl ether; dicetyl ether; polyglycerin-4 ethers; isoceteth-5; isoceteth-7, isoceteth-10; isoceteth-12; isoceteth-15; isoceteth-20; isoceteth-25; isoceteth-30; disodium lauroamphodipropionate; hexaethylene glycol monododecyl ether; and their mixtures; neopentyl glycol diisononanoate; cetearyl ethylhexanoate; panthenol ethyl ether, DL-panthenol, N-hexadecyl n-nonanoate, noctadecyl n-nonanoate, a profragrance, cyclodextrin, an encapsulation, and a combination thereof. At most 20% by weight, based on the total weight of the perfuming composition, of the modulator may be incorporated into the perfumed consumer product.

In the present description, the term ‘materialized’ is intended as existing outside of the digital environment of the present invention. ‘Materialized’ may mean, for example, readily found in nature or synthesized in a laboratory or chemical plant. In any event, a materialized composition presents a tangible reality. The terms ‘to be compounded’ or ‘compounding’ refer to the act of materialization of a composition, whether via extraction and assembly of ingredients or via synthetization and assembly of ingredients.

As used herein, the terms ‘computing system’ designate any electronic calculation device, whether unitary or distributed, capable of receiving numerical inputs and providing numerical outputs by and to any sort of interface, such as a digital interface. Typically, a computing system designates either a computer executing a software having access to data storage or a client-server architecture wherein the data and/or calculation is performed at the server side while the client side acts as an interface.

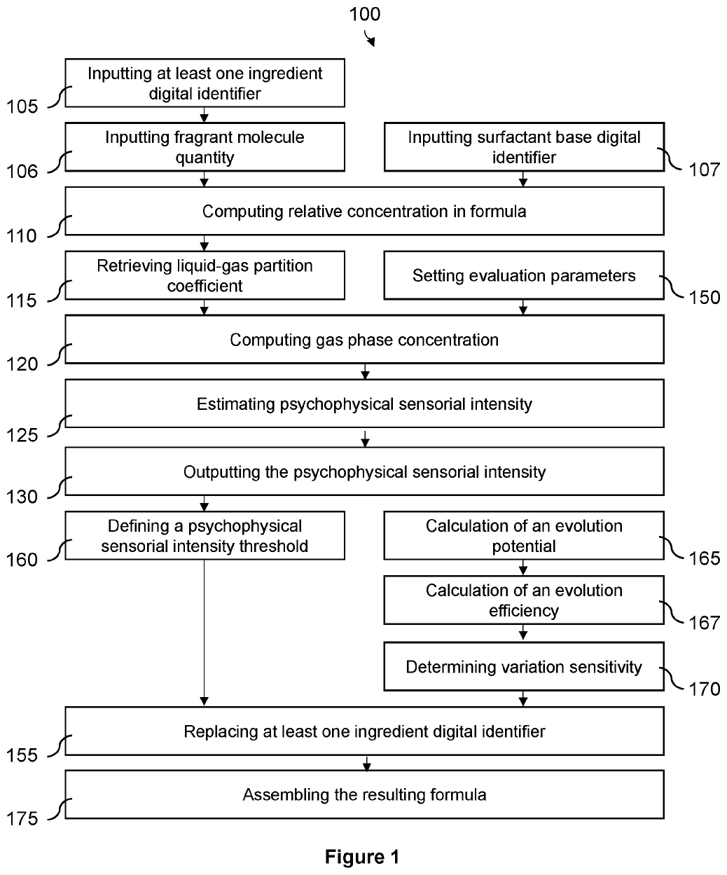

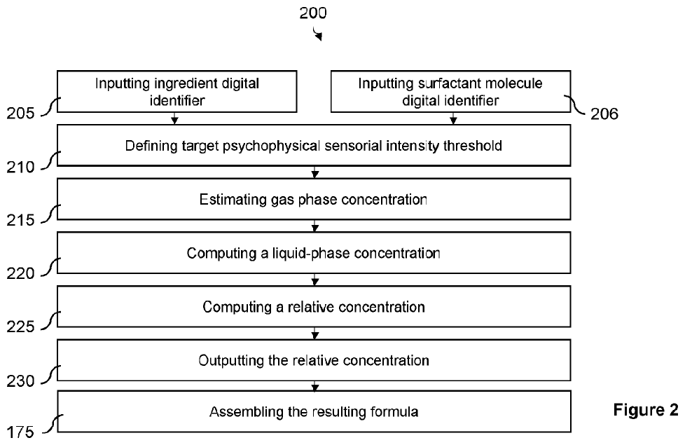

It should be noted that all steps that include computation may be run prior to the use of the results of said steps. These steps may alternatively be replaced by corresponding steps of extracting, from a computer memory, of the result of the computation. Such computations may be performed for specific input values corresponding to predetermined experimental conditions. The results of some of these steps may even be assimilated to constants. Nevertheless, for reasons of clarity and understanding of the concept of the present invention, FIGS. 1 and 2 show these steps of computation as being subsequent to one another.

It should be understood that one of the key advantages of the present invention is the capacity to predict realistic physical interactions and the resulting bloom performance in materialized formulas and in formulas to be materialized. Such advantages allow for the dynamic, efficient and fast reformulation of formulas based on the predicted performance of said formulas.

The inventors have discovered the following relations: The olfactive impact of blooming is in the first order related to the concentration of volatiles in the headspace having evaporated from an aqueous solution upon dilution with water as a function of time. The process of evaporation is governed by two independent partition coefficients: the micelle/water partition coefficient, K_{M}, and the water/air partition coefficient, K_{GL}. K_{M} is proportional to P_{O/W}, the n-octanol/water partition coefficient (mostly known as logP_{O/W}) and additionally depends on the nature of the surfactant, expressed by the so-called affinity factor (Colloids and Surfaces A 539, 2018, 310-318). At thermodynamic equilibrium, hydrophobic molecules such as fragrant molecules are distributed between the micellar phase, constituted by the surfactant molecules of the application base, and the water phase, respectively. K_{M} scales with the hydrophobicity of the molecules, and as a consequence the partitioning of the fragrance molecule is shifted from the water phase towards the micellar phase when the hydrophobicity of the molecule increases. The second partition coefficient, K_{GL}, is proportional to Henry's law constant and is specific for each individual fragrant molecule. It relates the gas phase concentration above an aqueous solution to the concentration of the volatile molecule in the liquid phase. Therefore, the gas phase concentration of a volatile depends directly on the micellar water partition coefficient, and as a consequence to the logP_{O/W} of the fragrant molecule and to the nature and the concentration of micellized surfactant molecules, respectively. Finally, the sensorial impact of a given fragrant molecule is related to its concentration in the gas phase under application conditions, where the perceived psychophysical sensorial intensity is a function of the dose-response curve of the particular fragrant molecule.

FIG. 1 shows a particular succession of steps of the method 100 object of the present invention. This aqueous composition sensorial impact determination method 100, comprises:
a step 105 of inputting at least one fragrant molecule digital identifier, upon a computer interface, said input defining a formula,
a step 106 of associating, for at least one input fragrant molecule digital identifier, a value representative of a quantity of the associated fragrant molecule to be input,
a step 107 of inputting at least one surfactant molecule digital identifier, upon a computer interface, said identifier being representative of a surfactant molecule in which the input surfactant molecule is organized in micelles, and where the input fragrant molecule partitions between the aqueous phase and the micellar phase of the surfactant molecule,
a step 110 of computing, by a computing device, a relative concentration of at least one fragrant molecule of the formula in the water phase and in the micellar phase formed by the corresponding surfactant as a function of the input formula and the associated quantity for at least one fragrant molecule digital identifier and the input surfactant molecule digital identifier,
a step 115 of retrieving, by a computing device, a liquid-gas partition coefficient of at least one said fragrant molecule,
a step 120 of computing, by a computing device, a gas phase concentration of at least one said fragrant molecule as a function of the liquid-gas partition coefficient and of the relative concentration in the water phase of said fragrant molecule,
a step 125 of estimating, by a computing device, a psychophysical sensorial intensity for at least one fragrant molecule of the formula as a function of the computed gas phase concentration and
a step 130 of outputting, upon a computer interface, the psychophysical sensorial intensity for at least one fragrant molecule of the formula.

It should be noted that the aqueous composition sensorial impact determination method can be understood as an aqueous composition sensorial impact simulation method. The objective of this method is to allow for the anticipation of the behavior of a fragrant molecule in application use conditions.

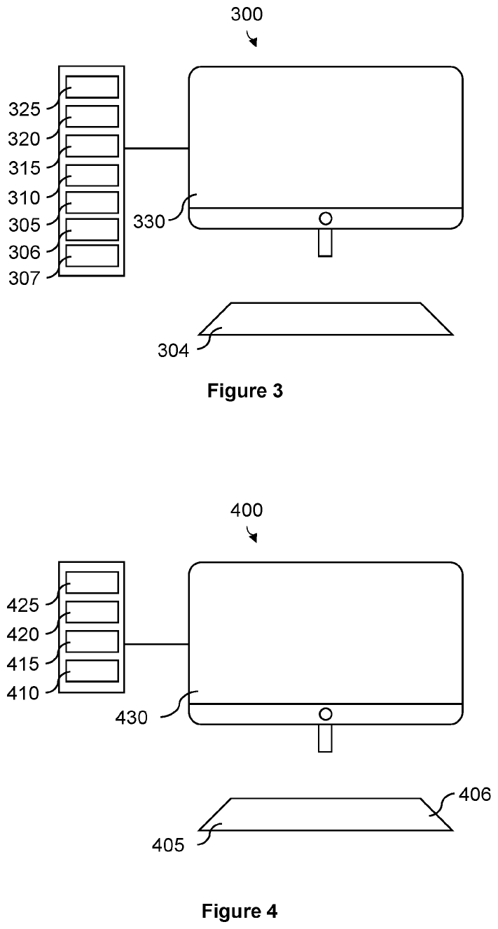

The step 105 of inputting is performed, for example, using any type of computer interface, such as a keyboard, a mouse or a touchscreen, for example, or a software controller, such as a controller 305 interacting with such a keyboard 304 such as represented in FIG. 3. Such interface may further comprise a graphical user interface (GUI) allowing for user interaction and input. This GUI may be part of a software ran by a computing means, such as a personal computer or computer server. In variants, the computer interface is logical in nature, the input corresponding to a command received through an electronic network or cable and originating from a command means. In such variants, the interface may be an application programming interface (API), for example.

The particular architecture of the computing system used in FIGS. 1 to 4 is unimportant with regards to the present invention. That is to say, such a computing system may be distributed, integrated, using a client-server architecture or using local and/or distant computing resources. Data stored and accessed may be stored in traditional databases, in computer memories or in distributed databases.

During this step of inputting 105, a user or program may select one or more fragrant molecule digital identifier to add in a formula. A fragrant molecule digital identifier may be an icon, a text label or a number for example. Such a fragrant molecule digital identifier corresponds, preferably, to an entry into a computer memory or a database.

A formula may further comprise a quantity of each fragrant molecule, expressed preferably in liquid phase quantities. Such a quantity may be expressed in parts per million (ppm) or in relative concentration of fragrant molecule in the total liquid quantity for example.

In particular embodiments, such as the one shown in FIG. 1, the method 100 comprises a step of inputting 106 a fragrant molecule quantity, either in absolute or relative values. Such a step of inputting 106 is functionally and structurally similar to any variant of the step of inputting 105.

The step of inputting 107 at least one surfactant molecule digital identifier functionally and structurally similar to any variant of the step of inputting 105.

The nature and type of the surfactant molecule will depend on the application. Non-limiting examples of suitable application may include a fabric care product, such as a liquid or solid detergent optionally in the form of a pod or tablet, a fabric softener, a liquid or solid scent booster, a dryer sheet, a fabric refresher, an ironing water, a paper, a bleach, a carpet cleaner, a curtain-care product; a body-care product, such as a hair care product (e.g. a shampoo, a leave-on or rinse-off hair conditioner, a coloring preparation or a hair spray, a color-care product, a hair shaping product, a dental care product), a disinfectant, an intimate care product; a cosmetic preparation (e.g. a skin cream or lotion, a vanishing cream or a deodorant or antiperspirant (e.g. a spray or roll on), a hair remover, a tanning or sun or after sun product, a nail product, a skin cleansing, a makeup); or a skin-care product (e.g. a soap, a shower or bath mousse, oil or gel, or a hygiene product or a foot/hand care products); an air care product, such as an air freshener or a “ready to use” powdered air freshener which can be used in the home space (rooms, refrigerators, cupboards, shoes or car) and/or in a public space (halls, hotels, malls, etc.); or a home care product, such as a mold remover, a furnisher care product, a wipe, a dish detergent or a hard-surface (e.g. a floor, bath, sanitary or a window-cleaning) detergent; a leather care product; a car care product, such as a polish, a wax or a plastic cleaner.

Such a surfactant molecule digital identifier may be, for example, but is not limited to:
a surfactant molecule that may be selected from the group of sodium C12-C15 pareth sulfate and cocamidopropyl betaine,
a surfactant molecule that comprises sodium laureth sulfate and cocamidopropyl betaine,
a surfactant molecule that comprises lauric acid, myristic acid, sodium laureth sulfate, and stearic acid,
a surfactant molecule that comprises sodium laureth sulfate, cocamidopropyl betaine and an alkylpolyglycoside,
a surfactant molecule that comprises ammonium lauryl sulfate, ammonium laureth sulfate, and cocamidopropyl betaine or
a surfactant molecule that comprises a linear alkylbenzene sulfonate, an ethoxylated fatty alcohol and sodium laureth sulfate.

In a particular embodiment of this step of inputting 107, a user or program may, upon a computer interface, select surfactant molecule digital identifier among a list of available surfactant molecules.

In a particular embodiment of this step of inputting 107, a user or program may, upon a computer interface, select an intended application for the formula, said application being associated to at least one surfactant molecule that is either automatically selected or prompted to the user or program for selection.

Such an application may be, for example, but is not limited to:
body care (liquid hand soap, shower gel, bar soap),
hair care (shampoo, conditioner),
surface care (all-purpose cleaner),
toilet care (rim blocks, liquid toilet cleaner, powder toilet cleaner),
dishwash liquid and
fabric care (liquid detergent, laundry bar, laundry powder)

Such applications may correspond to, for example, but is not limited to:
for a body care application, a surfactant molecule that may be selected from the group of sodium C12-C15 pareth sulfate and cocamidopropyl betaine,
for a body care application, a surfactant molecule that may be selected from the group consisting of sodium alkylether sulfate, ammonium alkylether sulfates, alkylamphoacetate, cocamide MEA, alkylglucosides and aminoacid based surfactants
for a body care application, a surfactant molecule that comprises sodium laureth sulfate and cocamidopropyl betaine,
for a body care application, a surfactant molecule that comprises lauric acid, myristic acid, sodium laureth sulfate, and stearic acid,
for a body care application, a surfactant molecule that comprises sodium laureth sulfate, cocamidopropyl betaine and an alkylpolyglycoside,
for a hair care application, a surfactant molecule that comprises ammonium lauryl sulfate, ammonium laureth sulfate, and cocamidopropyl betaine,
for a fabric care application, particularly softener, a surfactant molecule that may be selected from the group consisting of dialkyl quaternary ammonium salts, dialkyl ester quaternary ammonium salts, Hamburg esterquat, triethanolamine quat, silicones and mixtures thereof
for a fabric care application, particularly liquid detergent, a surfactant molecule that may be selected from the group consisting of alkylbenzenesulfonate, linear alkylbenzene sulfonates, secondary alkyl sulfonate, primary alcohol sulfate, lauryl ether sulfate, sodium lauryl ether sulfate, methyl ester sulfonate, alkyl amines, alkanolamide, fatty alcohol poly(ethylene glycol) ether, fatty alcohol ethoxylate , ethylene oxide and propylene oxide copolymers, amine oxydes, alkyl polyglucosides, alkyl polyglucosamides and mixtures thereof
for a fabric care application, particularly solid detergent, a surfactant molecule that may be selected from the group consisting of linear alkene benzene sulphonate, sodium laureth sulphate, sodium lauryl ether sulphate, sodium lauryl sulphate, alpha olefin sulphonate, methyl ester sulphonates, alkyl polyglyucosides, primary alcohol ethoxylates and in particular lauryl alcohol ethoxylates, primary alcohol sulphonates, soap and mixtures thereof, and
for a surface care application, a surfactant molecule that comprises a linear alkylbenzene sulfonate, an ethoxylated fatty alcohol and sodium laureth sulfate.

In particular embodiments, the method 100 object of the present invention comprises a step of inputting a quantity of at least one surfactant molecule, said quantity being used during the step of computing 110.

The step of computing 110 is performed, for example, by a computing system configured to run a dedicated software. During this step of computing 110, the objective is to determine the relative concentration of an ingredient in the water phase and in the micellar phase formed by the surfactant, respectively. Only the fraction in the water phase can eventually evaporate and contribute to blooming, the fraction within the micelles is not available. This relative concentration is known as the micelle-water partition coefficient, noted K_{M}. This coefficient may be determined by the formula:

$${K}_{M} = \frac{{c}_{micelle}}{{c}_{water phase}}$$

where:
c_{micelle} designates the concentration of an ingredient in the micellar phase and
c_{water phase} designates the concentration of an ingredient in the water phase. The higher the value for the micelle-water partition coefficient, the less fragrant molecules are available for evaporation and detection by the odorant receptors of the user.

In particular embodiments, such as shown in FIG. 1, this step of computing 110 is performed using the equation:

K_{M}=AF·P_{O/W}

where:
K_{M} is the micelle-water partition coefficient,
AF is an affinity factor and
P_{O/W} represents the octanol-water partition coefficient.

The affinity factor is related to the surfactant environment. Such an activity factor value may be obtained according to the method based on Self-diffusion Nuclear Magnetic Resonance (NMR) spectroscopy disclosed in the document “Competition between surfactants and apolar fragrances in micelle cores” (Colloids and Surfaces A 539 (2018) 310-318) published by Wolfgang Fieber, Sandy Frank, César Herrero. Sample values may be found in that document as well. This document is further included by reference in the content of this application.

P_{O/W} is a parameter that describes the polarity of organic molecules, where typically non-polar molecules possess higher P_{O/W} values than polar molecules. It is more commonly known in its logarithmic form (logP_{O/W}).

It is thus possible to determine the value of c_{water phase} from the other known values.

The step 115 of retrieving is performed, for example, by a computing system configured to run a dedicated software. During the step 115 of retrieving, the objective is to retrieve a value for a liquid-gas partition coefficient K_{GL} from a digital storage unit, such as a database or a hard drive. Also referred to as the dimensionless Henry constant. This coefficient may be calculated as such:

$${K}_{GL} = \frac{{c}_{gas}}{{c}_{liquid ( water phase )}}$$

### Where:

K_{GL} designates the Henry constant for a particular fragrant molecule,
c_{gas} designates the concentration of an ingredient in gas phase and
c_{liquid(water phase)} corresponds to c_{water phase} of the step 110 of computing.

The value for K_{GL} may be extracted from a database or computer memory of Henry constant values for sample fragrant molecules. Such a database corresponds, for example, to experimental databases, online databases or publications. In other embodiments, this value may be computed with appropriate software and stored within a database or computer memory. In another embodiment, the Henry constants can be computed with the program COSMOtherm.

Knowing both the Henry constant as well as the concentration of fragrant molecule in the water of the liquid phase, it is possible to calculate the gas phase concentration.

The step 120 of computing is performed, for example, by a computing system configured to run a dedicated software. During the step 120 of computing, the objective is to implement Fick's law of mass transfer kinetics from liquid to gas phase. The equation, corresponding to this law, adapted to the context of the present invention, may be:

$$\frac{dn}{dt} = {kA}_{GL} [ {c}_{L} ( t ) - {c}_{G} ( t ) / {K}_{GL} ]$$

Such as disclosed for example in the document “Mathematical Model of Flavor Release from Liquids Containing Aroma-Binding Macromolecules” in J. Agric. Food Chem. 1997, 45, 1883-1890 by authors Marcus Harrison and Brian P. Hills.

### Where:

$$\frac{dn}{dt}$$

liquid to the gas phase as a function of time,
c_{L}(t) corresponds to c_{water phase} as a function of time such as presented in regards of the step 110 of determination,
c_{G}(t) corresponds to the value for which the equation should be solved—namely gas phase concentration as a function of time.
k corresponds to a mass transfer constant, and
A_{GL} corresponds to the liquid surface area, that can be approximated to a constant in some variants.

Liquid volumes, air volumes, surfaces, dilution factor, time and other factors can all be adapted to a sensory protocol designed to assess the dynamic bloom performance.

In one embodiment, continuous dilution can be considered for in this invention, where the mass transfer equation can be calculated at 0.1 seconds time steps, and where each time dilution factor (corresponding to up to 15 L for 10 g of base, for example), mass transfer coefficient (from highly agitated to stagnant), and liquid surface area (from a watch glass to cover the entire shower tray) can be recalculated. The results may be stored within a computer memory.

As such, in particular embodiments, the step 120 of computing, by a computing device, a gas phase concentration is performed as a function of time, the psychophysical sensorial intensity being estimated as a function of said gas phase concentration.

In particular embodiments, such as shown in FIG. 1, the method 100 comprises a step 150 of setting, upon a computer interface, values of sensory evaluation parameters, such values being used in one of the steps upstream of the step of outputting.

Such evaluation parameters may be at least one of:
temperature of the water or the air (37° C., for example),
liquid volume of aqueous composition,
air volume or ambient air flow in which the fragrant molecule is transferred (1.6 m³, for example),
application surface area of the aqueous composition and optionally evolution over time (from 0.008 to 0.8m², for example),
agitation of the aqueous phase, expressed by a mass transfer coefficient, and optionally evolution over time, for example k=0.4×10⁻⁶ m/s for non-agitated or k=1×10⁻⁵ m/s for agitated)
dilution factor (1500, for example),
rate of addition of water (e.g., 10 L /min) and/or time intervals or total duration (10 s to 60 s from the input of water, for example).

Such evaluation parameters may target the simulation environment, the fragrance features and/or the surfactant molecule, for example. A surfactant molecule generally corresponds to an application base.

In particular embodiments, such as shown in FIG. 1, the method 100 comprises a step 150 of determination, by a computing device, of evaluation parameters as a function of a value representative of time since contact between the aqueous composition and a stream of water, the step 120 of computing a gas phase concentration being performed as a function of the liquid surface area determined.

The step 150 of determination is performed, for example, similarly to one of the variants of the step 105 of inputting. During this step 150 of determination, for example, a GUI may prompt a user to enter an initial value for the evaluation parameter to be used in the step 120 of computing as well as a final value at the end of the simulation. The evaluation parameter can then be determined via linear or polynomial interpolation, for example.

Alternatively, evaluation parameters may be computed using more advanced models from an initial value that is automatically or manually set. Such more advanced model may use fluid dynamics calculations, for example.

In another embodiment, instantaneous dilution can be considered for in this invention, comprising an initial dilution step followed by a process where the mass transfer equation can be calculated at various time steps, and where each time evaluation parameters are constant over time. The results may be stored within a computer memory.

In another embodiment the evaluation parameters are adjusted for evaluation in closed cabins.

In another embodiment the evaluation parameters are adjusted for evaluation in open cabins.

In another embodiment the evaluation parameters are adjusted for evaluation in cups.

In another embodiment the evaluation parameters are adjusted for evaluation in sinks.

In another embodiment the evaluation parameters are adjusted for evaluation in buckets.

In another embodiment the evaluation parameters are adjusted for evaluation on skin.

In another embodiment the evaluation parameters are adjusted for evaluation on hair swatches.

In another embodiment the evaluation parameters are adjusted for evaluation on hard surfaces.

In another embodiment the evaluation parameters are adjusted to represent the following applications, but is not limited to:
body care (liquid hand soap, shower gel, bar soap),
hair care (shampoo, conditioner),
surface care (all-purpose cleaner),
toilet care (rim blocks, liquid toilet cleaner, powder toilet cleaner),
dishwash and
fabric care (liquid detergent, laundry bar, laundry powder)

Such applications may correspond to those mentioned above.

The step 125 of estimating is performed, for example, by a computing system configured to run a dedicated software. During the step 125 of estimating, the gas phase concentration computed can be compared to existing psychophysical sensorial intensity, for said fragrant molecule, corresponding to this gas phase concentration.

In more advanced embodiments, this step 125 of estimating makes use of a dose-response curve. Such a dose-response curve is a mathematical formula (or the corresponding key parameters) defining the relationship between gas phase concentration and psychophysical sensorial intensity. Such a dose-response curve is typically sigmoidal and corresponds to a fit function between values corresponding to experimental results, obtained from panelists, for a particular predetermined gas phase concentration of a fragrant molecule.

The step 130 of outputting is performed, for example, using a computer screen and a graphic user interface (GUI) associated to a computer program designed to output such values. In other embodiments, the step 130 of outputting is performed using a data/digital output, such as an API or a communications network to provide the data estimated to another device or computer program.

In more advanced embodiments, such as the one shown in FIG. 1, the method 100 comprises a step 155 of replacing, by a computing device, at least one fragrant molecule digital identifier in the input formula as a function of the estimated psychophysical sensorial intensity of each said fragrant molecule and the estimated psychophysical sensorial intensity of at least one other fragrant molecule.

The step 155 of replacing is performed, for example, by a computing system configured to run a dedicated software. During the step 155 of replacing, several alternative or cumulative replacement criteria may be used.

Such a criterion may be, for example, the higher psychophysical sensorial intensity of a fragrant molecule than the input fragrant molecule currently in the formula.

Another criterion may be, for example, a similar psychophysical sensorial intensity, for a lower quantity than the input fragrant ingredient, of a fragrant molecule than the input fragrant molecule.

Another criterion may be, for example, tied to exogeneous to the fragrant molecule as such, such as dependent on the financial cost of said ingredient. In such a variant, a similar psychophysical sensorial intensity, for a lower financial cost than the input fragrant ingredient, of a fragrant molecule than the input fragrant molecule.

The step 155 of replacing is performed by executing the corresponding algorithm by the computing device upon a set of candidate fragrant molecules to determine whether said criterion is met. If so, the fragrant molecule digital identifier may be automatically changed to the new digital identifier of the fragrant molecule meeting said criterion. Alternatively, the new digital identifier of the fragrant molecule meeting said criterion may be output to a computing interface (GUI or API, for example), for manual or automatic third-party confirmation.

In variant, all candidate fragrant molecules for replacement are provided for confirmation or used for replacement. In other variants only one candidate fragrant molecule is provided for confirmation or used for replacement. Typically, the candidate fragrant molecule may be the one that has most met the criterion set.

In more advanced embodiments, such as the one shown in FIG. 1, the method 100 comprises a step 160 of defining, upon a computer interface, a psychophysical sensorial intensity threshold for at least one determined fragrant molecule digital identifier, the step 155 of replacing being performed as a function of the determined threshold.

In alternative embodiments, the step 155 of replacing is configured to provide an alternative quantity of a fragrant molecule already present in the formula, such alternative quantity being an increase or decrease, for example. Such an alternative quantity of a fragrant molecule is, in this scenario, an equivalent to a candidate fragrant molecule.

The step 160 of replacing is performed, for example, in a similar fashion (structurally and/or functionally) to the step of inputting 105. The set threshold can be used either as a minimum or as a maximum as a criterion to evaluate the potential for replacement.

As such, candidate fragrant molecule may then be algorithmically compared to the threshold and, if the criterion is met, be used as replacement or provided (upon a GUI for example) for confirmation of replacement.

In more advanced embodiments, such as the one shown in FIG. 1, the method 100 comprises a step 165 of calculation, by a computing device, of a psychophysical sensorial intensity evolution function of a fragrant molecule as a function of:
the gas phase concentration of said fragrant molecule and
a characteristic psychophysical sensorial intensity dose response curve linking gas phase concentration to psychophysical sensorial intensity,
   the step 155 of replacing being performed as a function of the psychophysical sensorial intensity evolution function of a fragrant molecule configured to be replaced and/or to replace another fragrant molecule in the formula.

The step 165 of calculation is performed, for example, by a computing system configured to run a dedicated software. During the step 165 of calculation the psychophysical sensorial intensity evolution function, called “bloom potential”, represents the ratio of the maximum gas phase concentration of a fragrant molecule that can be achieved in a given setup (i.e., as if the fragrant molecule was used at 100% in the formula) over the gas phase concentration needed to achieve a reference psychophysical sensorial intensity.

This bloom potential can be used as a criterion for replacement.

In particular embodiments, the method 100 object of the present invention further comprises optionally a step 170 of determining, by a computing device, a value representative of a sensitivity of variation of gas phase concentration at a reference point in the characteristic psychophysical sensorial intensity dose response curve of a fragrant molecule, the step 155 of replacing being performed as a function of the sensibility of a fragrant molecule configured to be replaced and/or to replace another fragrant molecule in the formula.

Such a reference point may be, for example, an inflexion point in the dose response curve or a predetermined reference point.

Such a value representative of sensitivity may correspond to an intensity slope.

Such a step 170 of determining may be performed, for example, by a dedicated software run upon a computing device.

In other embodiments, not represented, the method 100 object of the present invention comprises a step of determining a value representative of a delay to reach a reference psychophysical sensorial intensity for at least one fragrant molecule. Such a value may be measured in seconds or correspond to a relative ranking among a set of fragrant ingredients.

Such a step of determining is performed, for example, by a computing system configured to run a dedicated software. During this step of determining, the “intensity slope” of the dose response curve of a fragrant molecule may be used. The intensity slope is the slope of the dose response curve at the inflexion point of the sigmoidal function.

Alternatively, the “real time slope” of the dose response curve of a fragrant molecule may be used. The real time slope is the slope of the dose response curve at the actual gas phase concentration of a fragrant molecule that is obtained under application conditions.

This value representative of a delay to reach a reference psychophysical sensorial intensity can be used as a criterion for replacement.

In other embodiments, the method 100 object of the present invention comprises a step 167 of determining a value representative of the impact of increasing the quantity of a fragrant molecule in a formula on the final perceived intensity, called “bloom efficiency”. Bloom efficiency is the ratio of the increase in intensity over the increase in dosage to meet said intensity. The bloom efficiency may be calculated for several dosage variation increments or intensity variation increments.

Such a step 167 of determining is performed, for example, by a computing system configured to run a dedicated software.

This bloom efficiency can be used as a criterion for replacement.

Other criteria may be used to determine replacement fragrant molecule digital identifiers, such as the assessment of gas phase concentrations above various thresholds.

In such variants, the method 100 object of the present invention may comprise a step of computing, by a computing device, a gas phase concentration of at least one said fragrant molecule as a function of the liquid-gas partition coefficient and of the relative concentration in the water phase of said fragrant molecule. Such a gas phase concentration may then be compared to:
a value representative of the odor detection threshold for said fragrant molecule,
a value representative of the odor recognition threshold for said fragrant molecule,
a value representative of the gas phase concentration needed to achieve an intensity of 1.0 (on a scale between 0 and 6, where 0 represents no perceived intensity, and 6 represents the maximum perceived intensity of that compound),
a value representative of the gas phase concentration needed to achieve an intensity of 1.5 (on a scale between 0 and 6, where 0 represents no perceived intensity, and 6 represents the maximum perceived intensity of that compound),
a value representative of the gas phase concentration needed to achieve an intensity of 2.0 (on a scale between 0 and 6, where 0 represents no perceived intensity, and 6 represents the maximum perceived intensity of that compound),
a value representative of the gas phase concentration needed to achieve an intensity of 2.5 (on a scale between 0 and 6, where 0 represents no perceived intensity, and 6 represents the maximum perceived intensity of that compound),
a value representative of the gas phase concentration needed to achieve an intensity of 3.0 (on a scale between 0 and 6, where 0 represents no perceived intensity, and 6 represents the maximum perceived intensity of that compound),
a value representative of the gas phase concentration needed to achieve an intensity which is equal to the intensity at the inflexion point of the dose response curve.

A candidate for replacement may be obtained as a function of the result of the comparison for the above thresholds.

Other criteria may be used to determine replacement fragrant molecule digital identifiers, such as the overall bloom parameter for a fragrant molecule.

Such an overall bloom parameter (“Bloom Score”) for mixtures, or formulas, of fragrant ingredient may be defined as the sum of the gas phase concentration above one of the above-mentioned thresholds of all individual fragrant ingredients.

Such a bloom parameter may be defined in logarithmic form according to the following equation:

$$Bloom parameter = log ( {\sum }_{i = 1}^{n} {c}_{G}^{i} > threshold )$$

Or according to the following equation:

$$Bloom parameter = {\sum }_{I = 1}^{n} {c}_{G}^{i} \cdot {intensity}^{i} \cdot {slope}^{i}$$

In particular embodiments, the bloom parameter is dependent on the sensitivity of fragrant molecules with regards to variations in the gas phase concentrations of said fragrant molecules.

In such embodiments, the method 100 may further comprise a step of retrieving, by a computing device, the intensity slope of the dose response curve at the inflexion point of the sigmoidal function.

In such embodiments, the method 100 may further comprise a step of retrieving, by a computing device, the real-time slope of the dose response curve at the inflexion point of the sigmoidal function.

Other criteria may be used to determine replacement fragrant molecule digital identifiers, such as the classification of fragrant molecules into four groups according the following criteria:
(Group 1) Intensity of a fragrance molecule is above a reference intensity and intensity slope is above a reference intensity slope,
(Group 2) Intensity of a fragrance molecule is above a reference intensity and intensity slope is below a reference intensity slope,
(Group 3) Intensity of a fragrance molecule is below a reference intensity and intensity slope is above a reference intensity slope or
(Group 4) Intensity of a fragrance molecule is below a reference intensity and intensity slope is below a reference intensity slope.

Bloom performance of a fragrance is driven by the fragrant molecules in Group 1, followed by Group 3, Group 2 and Group 4, respectively. Therefore, in order to increase the bloom performance of a fragrance, it is desirable to increase the portion of fragrant molecules that are classified in Group 1. Alternatively, it can be desirable to increase the portion of other fragrant molecules that are classified in Group 2, Group 3, or Group 4, so that they reach either a reference intensity, or a reference slope, or both, in order to be classified in Group 1.

Therefore, a replacement criterion may be the belonging of a fragrant molecule to be replaced and the potential candidates to replace this fragrant molecule in particular groups.

It should be understood that the criteria used for one fragrant molecule can be used for the formula as a whole to evaluate the global bloom performance. This global bloom performance can be compared to global performance criteria (similar to fragrant molecule performance criteria discussed above). If the formula does not meet said criteria, the method may trigger the replacement of at least one constitutive fragrant ingredient.

FIG. 2 shows, schematically, a particular embodiment of the method 200 object of the present invention. This aqueous composition ingredient quantity determination method 200, comprises:
a step 205 of inputting at least one fragrant molecule digital identifier, upon a computer interface, said input defining a formula,
a step 206 of inputting at least one surfactant molecule digital identifier, upon a computer interface, said identifier being representative of a surfactant molecule in which the input surfactant molecule is organized in micelles, and where the input formula partitions between the aqueous phase and the micellar phase of the surfactant molecule,
a step 210 of defining, upon a computer interface, a value of target psychophysical sensorial intensity for at least one fragrant molecule of the formula,
a step 215 of estimating, by a computing device, a gas phase concentration for at least one fragrant molecule of the formula as a function of the defined target psychophysical sensorial intensity,
a step 220 of computing, by a computing device, a liquid-phase concentration of at least one said fragrant molecule as a function of the estimated gas phase concentration of said fragrant molecule,
a step 225 of computing, by a computing device, a relative concentration of at least one fragrant molecule of the formula in the water phase and in the micellar phase formed by the corresponding surfactant as a function of the liquid-phase concentration computed and
a step 230 of outputting, upon a computer interface, the relative concentration of at least one fragrant molecule of the formula.

This method corresponds to a reverse use of the teachings of the method 100 shown in FIG. 1. As such, the constitutive steps are structurally and/or functionally identical to the variants of their FIG. 1 counterpart. Furthermore, all variants and particular embodiments of FIG. 1 may also be implemented regarding this method 200.

In particular embodiments of the present invention, the method, 100 and/or 200, further comprises a step 175 of assembling the formula resulting from said method.

Such a step 175 of assembling may be performed by any means used to assemble chemical formulas. Such means may be, for example, a laboratory or a chemical manufacturing plant, for example.

FIG. 3 shows, schematically, a particular embodiment of the system 300 object of the present invention. This aqueous composition sensorial impact determination system 300, comprises:
a means 305 for inputting at least one fragrant molecule digital identifier, upon a computer interface, said input defining a formula,
a means 307 of associating, for at least one input fragrant molecule digital identifier, a value representative of a quantity of the associated fragrant molecule to be input,
a means 306 for inputting at least one surfactant molecule digital identifier, upon a computer interface, said identifier being representative of a surfactant molecule in which the input surfactant molecule is organized in micelles, and where the input fragrant molecule partitions between the aqueous phase and the micellar phase of the surfactant molecule,
a means 310 for computing, by a computing device, a relative concentration of at least one fragrant molecule of the formula in the water phase and in the micellar phase formed by the corresponding surfactant as a function of the input formula and the associated quantity for at least one fragrant molecule digital identifier and the input surfactant molecule digital identifier,
a means 315 for retrieving a liquid-gas partition coefficient of at least one said fragrant molecule,
a means 320 for computing a gas phase concentration of at least one said fragrant molecule as a function of the liquid-gas partition coefficient and of the relative concentration in the water phase of said fragrant molecule,
a means 325 for estimating a psychophysical sensorial intensity for at least one fragrant molecule of the formula as a function of the computed gas phase concentration and
a means 330 for outputting, upon a computer interface, the psychophysical sensorial intensity for at least one fragrant molecule of the formula.

Particular embodiments and implementation possibilities of the means of the system 300 have been disclosed in regard to FIG. 1. As such, the means 305 for inputting may be a GUI associated to a dedicated software or an API. The means for computing 310, for retrieving 315, for computing and for estimating 325 may be, for example, dedicated software running upon an electronic circuit, such as a computing device. This computing device may be local or distant.

FIG. 4 shows, schematically, a particular embodiment of a system 400 object of the present invention. The aqueous composition ingredient quantity determination system 400, comprises:
a means 405 for inputting at least one fragrant molecule digital identifier, upon a computer interface, said input defining a formula,
a means 406 for inputting at least one surfactant molecule digital identifier, upon a computer interface, said identifier being representative of a surfactant molecule in which the input surfactant molecule is organized in micelles, and where the input formula partitions between the aqueous phase and the micellar phase of the surfactant molecule,
a means 410 for defining a value of target psychophysical sensorial intensity for at least one fragrant molecule of the formula,
a means 415 for estimating a gas phase concentration for at least one fragrant molecule of the formula as a function of the defined target psychophysical sensorial intensity,
a means 420 for computing a liquid-phase concentration of at least one said fragrant molecule as a function of the estimated gas phase concentration of said fragrant molecule,
a means 425 for computing a relative concentration of at least one fragrant molecule of the formula in the water phase and in the micellar phase formed by the corresponding surfactant as a function of the liquid-phase concentration computed and
a means 430 for outputting, upon a computer interface, the relative concentration of at least one fragrant molecule of the formula.

Similarly, to FIG. 2, the system 400 of FIG. 4 corresponds to a particular use of the constitutive means and steps of both the system 300 of FIG. 3 and the method 200 of FIG. 2. The constitutive means of this system 400 are thus similar to the means of the system 300.

1. Aqueous composition sensorial impact determination method (100), said method comprising:
a step (105) of inputting at least one fragrant molecule digital identifier, upon a computer interface, said input defining a formula,
a step (106) of associating, for at least one input fragrant molecule digital identifier, a value representative of a quantity of the associated fragrant molecule to be input,
a step (107) of inputting at least one surfactant molecule digital identifier, upon a computer interface, said identifier being representative of a surfactant molecule in which the input surfactant molecule is organized in micelles, and where the input fragrant molecule partitions between the aqueous phase and the micellar phase of the surfactant molecule,
a step (110) of computing, by a computing device, a relative concentration of at least one fragrant molecule of the formula in the water phase and in the micellar phase formed by the corresponding surfactant as a function of the input formula and the associated quantity for at least one fragrant molecule digital identifier and the input surfactant molecule digital identifier,
a step (115) of retrieving, by a computing device, a liquid-gas partition coefficient of at least one said fragrant molecule,
a step (120) of computing, by a computing device, a gas phase concentration of at least one said fragrant molecule as a function of the liquid-gas partition coefficient and of the relative concentration in the water phase of said fragrant molecule,
a step (125) of estimating, by a computing device, a psychophysical sensorial intensity for at least one fragrant molecule of the formula as a function of the computed gas phase concentration and
a step (130) of outputting, upon a computer interface, the psychophysical sensorial intensity for at least one fragrant molecule of the formula.

2. Method (100) according to claim 1, which further comprises a step (150) of setting, upon a computer interface, values of sensory evaluation parameters representative of at least one of:
temperature of the water or the air,
liquid volume of aqueous composition,
air volume into which the fragrant molecule is transferred,
application surface of the aqueous composition and evolution over time,
dilution factor,
application surface area,
rate of addition of water,
agitation of aqueous phase and/or
ambient air flow,
such values being used at least one of the steps upstream of the step (130) of outputting.

3. Method (100) according to claim 1, in which the step (120) of computing, by a computing device, a gas phase concentration is performed as a function of time, the psychophysical sensorial intensity estimated being determined as a function of said gas phase concentration.

4. Method (100) according to claim 1, in which the step (110) of computing is performed using the equation:
K_{M}=AF·P_{O/W}
where:
K_{M} is the micelle-water partition coefficient of the fragrant molecules between micellar and aqueous phases,
AF is an affinity factor,
P_{O/W} represents the octanol-water partition coefficient.

5. Method (100) according to claim 1, which further comprises a step (150) of determination, by a computing device, of evaluation parameters as a function of a value representative of time since contact between the aqueous composition and a stream of water, the step (120) of computing a gas phase concentration being performed as a function of the evaluation parameter determined.

6. Method (100) according to claim 1, which comprises a step (155) of replacing, by a computing device, at least one fragrant molecule digital identifier in the input formula as a function of the estimated psychophysical sensorial intensity of each said ingredient and the estimated psychophysical sensorial intensity of at least one other fragrant molecule.

7. Method (100) according to claim 6, which comprises a step (160) of defining, upon a computer interface, a psychophysical sensorial intensity threshold for at least one determined fragrant molecule digital identifier, the step (155) of replacing being performed as a function of the determined threshold.

8. Method (100) according to claim 6, which comprises a step (165) of calculation, by a computing device, of a psychophysical sensorial intensity evolution function of a fragrant molecule as a function of:
the gas phase concentration of said fragrant molecule and
a characteristic psychophysical sensorial intensity dose response curve linking gas phase concentration to psychophysical sensorial intensity, the step (155) of replacing being performed as a function of the psychophysical sensorial intensity evolution function of a fragrant molecule configured to be replaced and/or to replace another fragrant molecule in the formula.

9. Method (100) according to claim 8, which further comprises a step (170) of determining, by a computing device, a value representative of a sensitivity of variation of gas phase concentration at a reference point in the characteristic psychophysical sensorial intensity dose response curve of a fragrant molecule, the step (155) of replacing being performed as a function of the sensibility of a fragrant molecule configured to be replaced and/or to replace another fragrant molecule in the formula.

10. Aqueous composition ingredient quantity determination method (200), said method comprising:
a step (205) of inputting at least one fragrant molecule digital identifier, upon a computer interface, said input defining a formula,
a step (206) of inputting at least one surfactant molecule digital identifier, upon a computer interface, said identifier being representative of a surfactant molecule in which the input surfactant molecule is organized in micelles, and where the input formula partitions between the aqueous phase and the micellar phase of the surfactant molecule,
a step (210) of defining, upon a computer interface, a value of target psychophysical sensorial intensity for at least one fragrant molecule of the formula,
a step (215) of estimating, by a computing device, a gas phase concentration for at least one fragrant molecule of the formula as a function of the defined target psychophysical sensorial intensity,
a step (220) of computing, by a computing device, a liquid-phase concentration of at least one said fragrant molecule as a function of the estimated gas phase concentration of said fragrant molecule,
a step (225) of computing, by a computing device, a relative concentration of at least one fragrant molecule of the formula in the water phase and in the micellar phase formed by the corresponding surfactant as a function of the liquid-phase concentration computed and
a step (230) of outputting, upon a computer interface, the relative concentration of at least one fragrant molecule of the formula.

11. Method (100, 200) according to claim 1, which further comprises a step (175) of assembling the formula resulting from said method.

12. Aqueous composition sensorial impact determination system (300), wherein said system comprises:
a means (305) for inputting at least one fragrant molecule digital identifier, upon a computer interface, said input defining a formula,
a means (307) of associating, for at least one input fragrant molecule digital identifier, a value representative of a quantity of the associated fragrant molecule to be input,
a means (306) for inputting at least one surfactant molecule digital identifier, upon a computer interface, said identifier being representative of a surfactant molecule in which the input surfactant molecule is organized in micelles, and where the input fragrant molecule partitions between the aqueous phase and the micellar phase of the surfactant molecule,
a means (310) for computing, by a computing device, a relative concentration of at least one fragrant molecule of the formula in the water phase and in the micellar phase formed by the corresponding surfactant as a function of the input formula and the associated quantity for at least one fragrant molecule digital identifier and the input surfactant molecule digital identifier,
a means (315) for retrieving a liquid-gas partition coefficient of at least one said fragrant molecule,
a means (320) for computing a gas phase concentration of at least one said fragrant molecule as a function of the liquid-gas partition coefficient and of the relative concentration in the water phase of said fragrant molecule,
a means (325) for estimating a psychophysical sensorial intensity for at least one fragrant molecule of the formula as a function of the computed gas phase concentration and
a means (330) for outputting, upon a computer interface, the psychophysical sensorial intensity for at least one fragrant molecule of the formula.

13. Aqueous composition ingredient quantity determination system (400), said system comprising:
a means (405) for inputting at least one fragrant molecule digital identifier, upon a computer interface, said input defining a formula,
means (406) for inputting at least one surfactant molecule digital identifier, upon a computer interface, said identifier being representative of a surfactant molecule in which the input surfactant molecule is organized in micelles, and where the input formula partitions between the aqueous phase and the micellar phase of the surfactant molecule,
a means (410) for defining, upon a computer interface, a value of target psychophysical sensorial intensity for at least one fragrant molecule of the formula,
a means (415) for estimating a gas phase concentration for at least one fragrant molecule of the formula as a function of the defined target psychophysical sensorial intensity,
a means (420) for computing a liquid-phase concentration of at least one said fragrant molecule as a function of the estimated gas phase concentration of said fragrant molecule,
a means (425) for computing a relative concentration of at least one fragrant molecule of the formula in the water phase and in the micellar phase formed by the corresponding surfactant as a function of the liquid-phase concentration computed and
a means (430) for outputting, upon a computer interface, the relative concentration of at least one fragrant molecule of the formula.

14. Method (100) according to claim 7, which comprises a step (165) of calculation, by a computing device, of a psychophysical sensorial intensity evolution function of a fragrant molecule as a function of:
the gas phase concentration of said fragrant molecule and
a characteristic psychophysical sensorial intensity dose response curve linking gas phase concentration to psychophysical sensorial intensity, the step (155) of replacing being performed as a function of the psychophysical sensorial intensity evolution function of a fragrant molecule configured to be replaced and/or to replace another fragrant molecule in the formula.

15. Method (100) according to claim 14, which further comprises a step (170) of determining, by a computing device, a value representative of a sensitivity of variation of gas phase concentration at a reference point in the characteristic psychophysical sensorial intensity dose response curve of a fragrant molecule, the step (155) of replacing being performed as a function of the sensibility of a fragrant molecule configured to be replaced and/or to replace another fragrant molecule in the formula.
